(19) United States
(12) Patent Application Publication
(10) Pub. No.: US 2018/0064424 A1
(43) Pub. Date: Mar. 8, 2018

(54) URINE SPECIMEN COLLECTION SYSTEM

(71) Applicant: ORUBA MEDIKAL TEKNOLOJI ARGE IC VE DIS TIC. LTD. STI, Ankara (TR)

(72) Inventor: Utku ULUSAHIN, Ankara (TR)

(73) Assignee: ORUBA MEDIKAL TEKNOLOJI ARGE IC VE DIS TIC. LTD. S TI., Ankara (TR)

(21) Appl. No.: 15/561,513

(22) PCT Filed: Mar. 23, 2016
(86) PCT No.: PCT/TR2016/050075

§ 371 Date: Sep. 25, 2017

(30) Foreign Application Priority Data
Mar. 25, 2015 (TR) 2015/03588
Apr. 17, 2015 (TR) 2015/04747
Mar. 8, 2016 (TR) 2016/03058
Mar. 15, 2016 (TR) 2016/03360

(51) Int. Cl. A61B 10/00; A61B 5/20; G01F 9/00

(52) U.S. Cl. CPC A61B 10/007; G01F 9/003; A61B 5/208

(57) ABSTRACT

The present invention relates to an urine specimen collection system in which the urine sample to be taken for the laboratory tests, can be collected without interrupting the patient while urinating. Present invention will transfer certain amount of urine into the specimen container by collecting the urine of the patient collected during the urination speed test. Thus, the specimen acquired from the urination speed test can be used in the laboratory tests. System will transfer the urine of the patient into a specimen container automatically by steps applied during the regular urination process and immediately after this process. System then will perform the cleaning of the urinated reservoir.

### CROSS REFERENCE TO RELATED APPLICATIONS

This application is the national phase entry of International Application No. PCT/TR2016/050075, filed on Mar. 23, 2016, which is based upon and claims priority to Turkish Patent Application No. 2015/03588 (TR), filed on Mar. 25, 2015, and Turkish Patent Application No. 2015/04747 (TR), filed on Apr. 17, 2015, and Turkish Patent Application No. 2016/03058 (TR), filed on Mar. 8, 2016, and Turkish Patent Application No. 2016/03360 (TR), filed on Mar. 15, 2016, the entire contents of which are incorporated herein by reference.

### TECHNICAL FIELD

The present invention relates to a urine specimen collection system in which urine samples to be taken for the laboratory tests, can be collected without interrupting the person while urinating.

### BACKGROUND

Urine can be considered as a sterile body secretion for a healthy individual. The Chemical structure of the urine that is formed as a result of the filtration of toxins and metabolic wastes in the body by the kidneys is altered because of infections and physical degradations occurring in the urinary system. Because urinary test results enable the physicians to gain insights about many issues, it is the second most desired test by the physicians.

In order to perform the test, urine samples are demanded from patients in a specimen container. Transporting the specimen to be examined in the laboratory, in-to a urine specimen container will be performed by the patient. Therefore, the patient has to urinate into a container which the patient also holds by hand. This unhygienic situation can also compromise the hygiene of the surrounding environment. At the same time, in order to obtain accurate test results, the urine sample collection process should be hygienic. An example of urine sample collector is disclosed in United Kingdom patent application GB2162312A. In this application, according to the principle of computational fluid, an individual transports his/her urine into a container through a funnel. Then, filling of urine into a specimen container which has the same base height, occurs. The obtained specimen tube is removed by pulling the obtained specimen tube out from the center of the base and the obtained specimen is transported to a test tube via a syringe. The collection of urine sample into the container is performed by individuals.

At the same time, in certain conditions, the urination speeds of patients required to submit urine samples is also measured for the diagnosis and diagnosis can be made with the help of obtained results. An example of aforementioned processes known in the field is disclosed in patent application US2012226196. In this application, a single-use, inexpensive and disposable container is mentioned. With the help of this aforementioned container, the patients are directed during the urination and can cast away the container later.

Another example known in the field is disclosed in another United States patent application US2005256428. In this aforementioned application, the weight of the urine is measured gravimetrically during urination. With the help of this aforementioned measurement, the urination speed can be measured at the same time.

### SUMMARY OF THE INVENTION

The present invention relates to a urine collection system in which the required urine specimen is collected during the urination speed measurement. The present invention is a system that can automatically transport the required amount of urine from the container in which the urine is collected for urination speed measurement to urine specimen container, during the urination process for urination speed measurement.

The system which will include a contemporary pissoir and/or water closet like urination reservoir collects the inbound urine from this reservoir into a measuring container. The data obtained from the differences of the reservoir weight dynamically is used to calculate the urination speed. Then during the cleaning process, the required amount of urine located in the measuring container is transported into the urine specimen container and thus, the required urine for the laboratory analysis is obtained. Then, system performs cleaning stages and will be ready for the next patient.

Through the usage of both water and incrementally disinfectant materials in the aforementioned cleaning process, the present invention provides the hygiene of the system and increased cabin service life.

### BRIEF DESCRIPTION OF THE DRAWINGS

Urine Specimen Collection System established for fulfilling the purposes of the present invention is shown in the accompanying figures, in which;

FIG. 1—Schematic illustration of the subject of the present invention, urine specimen collection system.

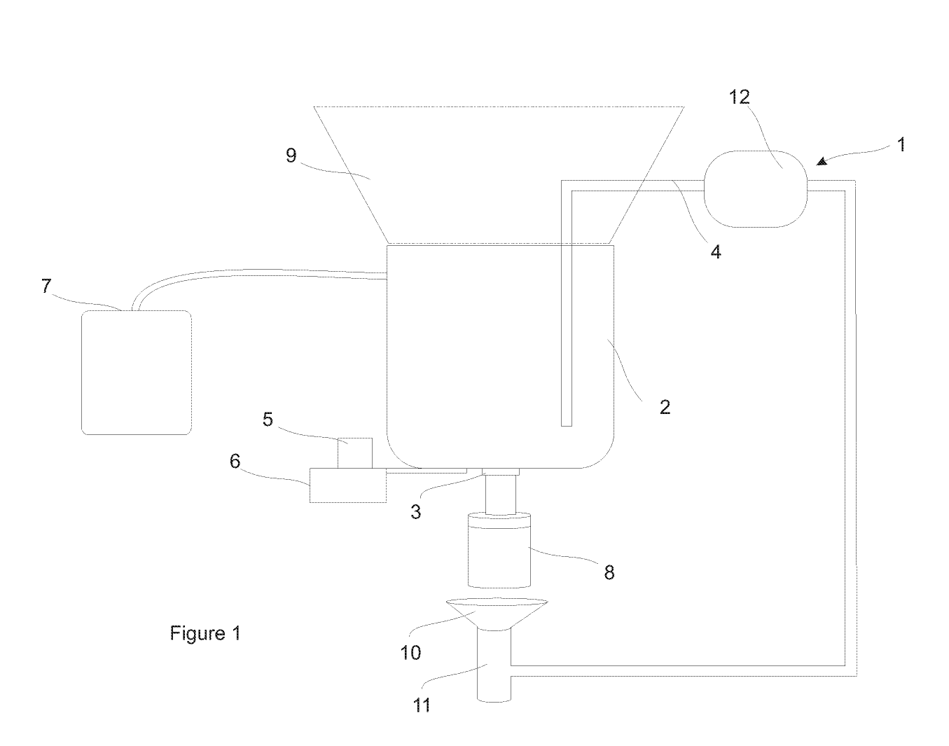

FIG. 2—Schematic illustration of Drain valve containing version of the subject of the present invention, urine specimen collection system.

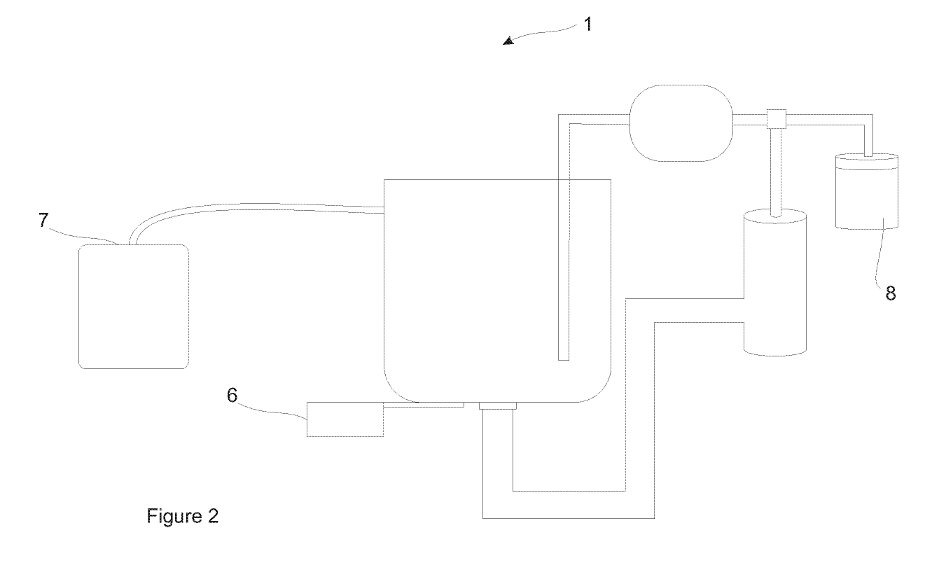

FIG. 3—Schematic illustration of single drain down version of the subject of the present invention, urine specimen collection system.

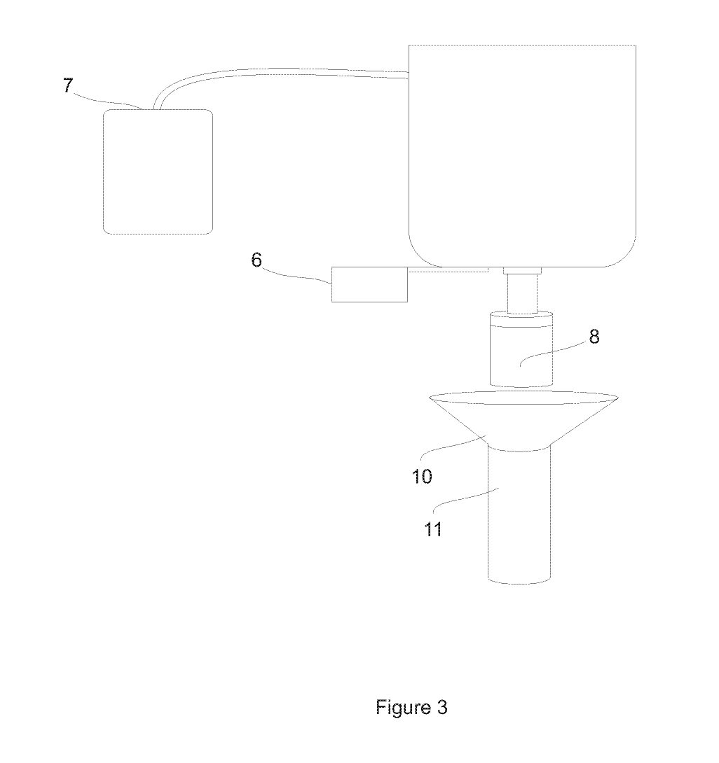

FIG. 4—Working algorithm of the subject of the present invention, urine specimen collection system.

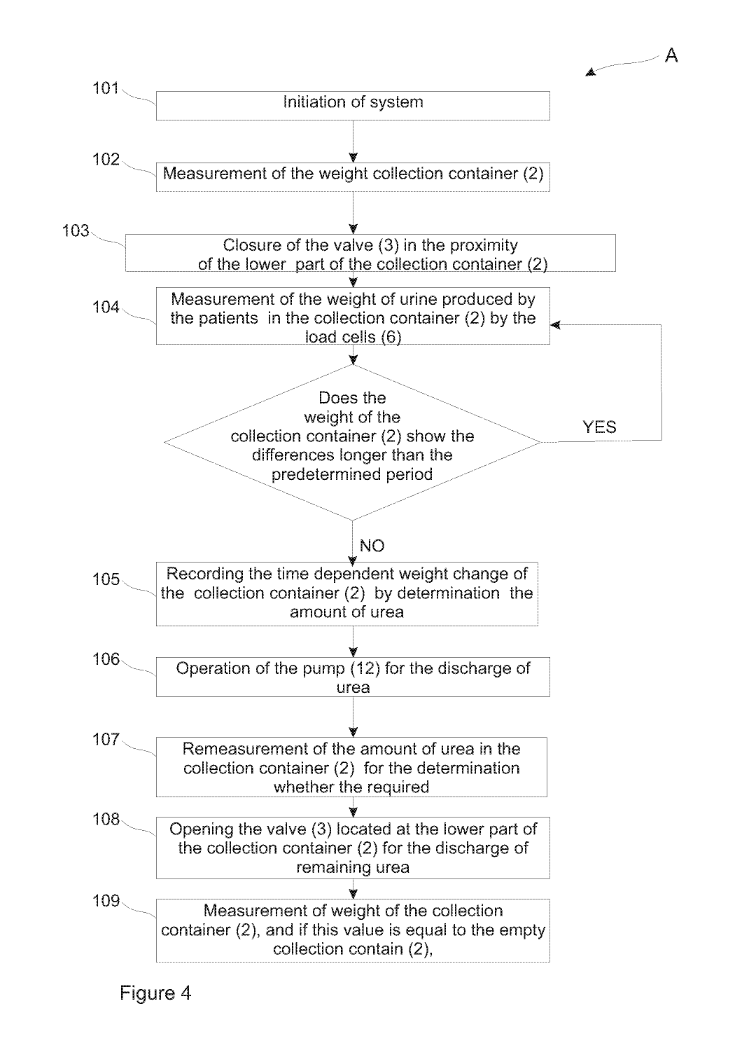

FIG. 5—Cleaning algorithm of the subject of the present invention, urine specimen collection system.

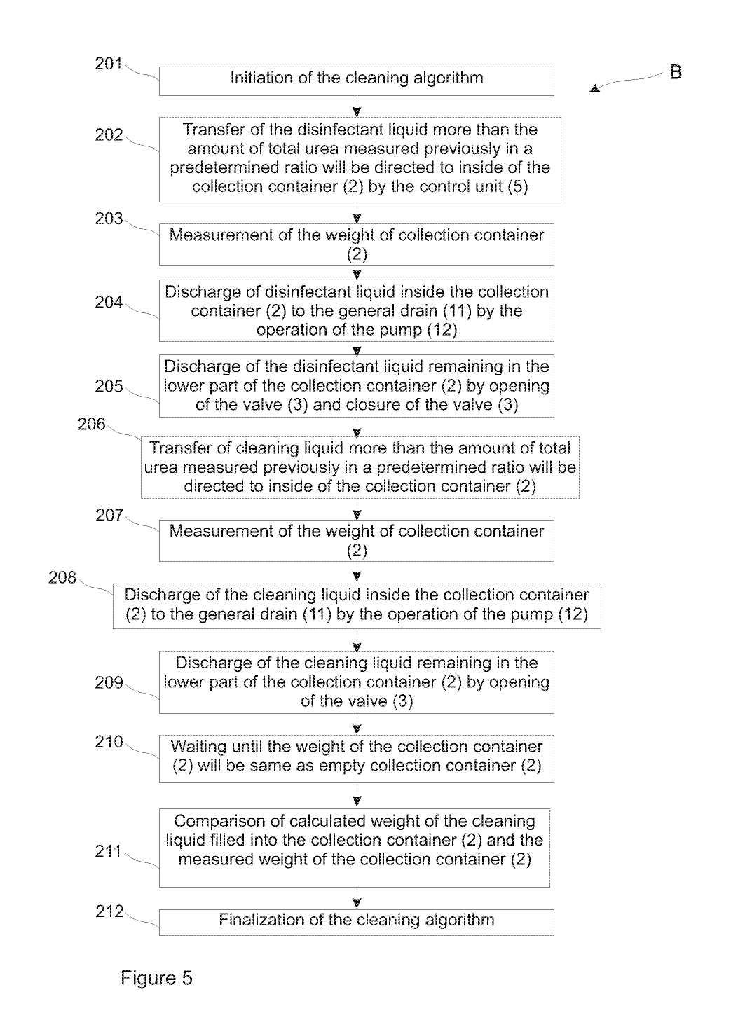

Parts in figures are numbered and the provisions of the numbers are given below.
1. Urine sample collection system
2. Collection container
3. Valve
4. Draining Pipe
5. Control unit
6. Load Cell
7. Tank
8. Specimen container
9. Reservoir
10. Funnel
11. General drain
12. Pump
A=Working Algorithm
B=Cleaning Algorithm.

### DETAILED DESCRIPTION OF THE EMBODIMENTS

Urine specimen collection system (1), subject of the present invention, consists of a collection container (2), a valve (2), a load cell (6), a pump (12), a specimen container (8) and a control unit (5) which records the eight information from the load cell (6) and determines the opening tune of the valve in its principal form. While the patient to be analyzed urinates into the reservoir (9), urea produced by patient is directed into the collection container (2) and collected there. During this interval, weight of the collection container (2) is measured by load cell (6) dynamically, measurement values are recorded by control unit (5). Thus, with the finalization of urine flow of the patient to be analyzed, the transfer of the collected urine into collection container (2) via the general drain (11) and drain pipe (4) is accomplished when the urine flow of the patient finishes and activates pump (12) by the control unit (5). After this disposal process, valve (3) located under the collection container (2) opens, and urea remaining in the collection container (2) is transferred into the specimen container (8). Then, cleaning processes are performed by the control unit (5). Therefore, with the completion of all these steps, a urine sample is collected and the urination speed of the patient is also recorded/taken/measured.

The time dependent graphic of weight increase recorded by the load cell (6) according to the urination speed of the patient, can be generated by the control unit (5). By the virtue of aforementioned information, gravimetrical measurement of flow rate is accomplished.

In order to compare the accuracy of the measurements of the load cell (6) that performed gravimetric measurement, a liquid with known density is employed. Liquid, stored in the aforementioned liquid tank (7) and used for cleaning of the remaining urea residues in the collection container (2) may be water, antiseptic or disinfectant liquid. Besides, it is also possible that rather than having liquid supplied from the tank (7), liquid can also be directly supplied from the municipal water system. Calibration of the system can be accomplished by taking the liquid mentioned above and filling a container with a constant velocity and measuring the filling speed and total weight recorded by the load cell (6).

In order for the present invention to complete the aforementioned processes, control unit (5) is present on the invention. Aforementioned control unit (5) can measure the weight of the collection container (2) with the load cell (6) and can switch the valve (3). Control unit (5) can also control pump (12) for cleaning processes. Control unit (5) also performs the aforementioned calibration. Control unit (5) operates these components whose mode of operation is disclosed previously here, according to a working algorithm (A).

Collection container (2) is where the urine of patients is collected in urine specimen collection system (1). There is a valve (3) controlled by the control unit, beneath the collection container (2). In different embodiments of the invention, this valve (3) is used for the transfer of the urine in the collection container (2) to specimen container (8) or general drain (11). Additionally, during the cleaning process, the cleaning liquid in collection container (2) is discharged with this valve (3). The load cell (6) located beneath collection container (2) measures the weight of collection container (2). Obtained information is recorded by transferring to control unit (5). Thus, the dynamically measurement of the patients urine flow is provided.

Draining pipe (4) is in a slouched configuration in collection container (2). Through this draining pipe (4), the patient's urine in collection container (2) is disposed after the urination speed measurement processes. Besides, in different embodiments of the present invention, the draining pipe (4) is used for disposal of the cleaning liquid. Urea or the cleaning liquid inside the collection container (2) will be directed to general drain (11) or specimen container (8) with the help of a pipe (12). In the pumped disposal version of the present invention, since the transfer of the urine specimen in the collection container (2) to the specimen container (8) is performed through the valve (3) located beneath of the collection container (2), the draining pipe (4) will located higher than the base of the collection container (2). With this height, volume of the cylinder formed the base surface of the collection container (2) will be smaller than volume of the specimen container (8). Thus, when the draining pipe (4) discharge the urine located in the collection container (2) with the help of the pump (12), it is provided that the amount of urea required to fill the specimen container (8) will be left in the collection container (2).

There will be (1) a tank (7) filled with disinfectant liquid in the urine specimen collection system for the functioning of cleaning algorithm (B). This tank (7) can be connected to municipal water supply. Thus, when the necessary measurement processes are completed, system (1) can use the liquid inside the tank (7) for self-cleaning. Additionally, since the amount water used for cleaning in the tank (7) is known, this information can be used in the calibration of urine specimen collection system.

When the urine collection system (i) working algorithm (A) is started (101), system will measure the weight of the collection container (2) (102). Weight of the collection container (2) can be different than the prespecified value because of solid or liquid residues remaining on the collection container (2). Thus, the weight of the collection container should be measured before every use. It is provided that this measurement value will be the reference weigh value in the control unit (5). Closure of the valve (3) located in the proximity of the base of the collection container (2) will be provided for urine flow rate measurements can be performed on the urine collected in to the collection container (2) (103). The audio and/or visual warning for noticing the patients that the system is ready for operation can be applied at this stage.

The amount of urea collected by the direction of patients urine in to the collection container (2) will be measured by the load cell (6) (104). Adjustment of the frequency of weight information gathered by the load cell (6) and the time dependent weight change of the collection container (2) will be governed by the control unit (5). Time dependent weight measurements will be performed on the collection container (2) until there won't be any change in weight of the predetermined time frame. If more time passes than the predetermined period without a change in weight, it is assumed the patient's urination is ceased. Then, it is possible than the user can be notified by audio and/or illuminated way about the measurement process is over or there won't be any new measurements taken.

In order to calculate the collected amount of urea accumulated into the collection container (2), the difference between first and last weight of the container (2) and the time dependent weight increase of the container (2) will be recorded (105). Thus, time dependent change in the urination speed of the patient is determined and required information for urination speed analysis is recorded. After the recording processes, pump will be activated for the discharge of the urea first (106). Thus, urea located in the container will be discharged to general drain (11) with the help of the pump (12) through the draining pipe (4). Since the draining pipe (4) is in a slouched configuration standing higher than the base of the collection container (2), it is expected that there will be urea in this volume left in the collection container (2). By measuring the collection container (2) again by the load cell (6), it is determined that whether the remaining volume of urea left in the container (2) is suitable for the specimen container (8) (107). Then, by opening of the valve (3) located beneath the collection container (2), remaining urea in the low end of the collection container (2) will be transferred to specimen container (8) (108). After, waiting for a predetermined period for transfer of urea into the specimen container (8), the weight of the collection container (2) will be measured again and it is ensured that little or any urea left inside and valve(3) will be closed prior to the cleaning process (108).

Hereinabove, working algorithm (A) of the urine specimen collection system's (1) during the measurements taken from the patient is described in detail. Nonetheless, after every time it is used it is required both for hygiene and accuracy of measurements that the urine specimen collection system (1) is become ready. For this purpose cleaning algorithm (B) will be operated (201).

Cleaning algorithm (B) is required for the discharge of residual urea and sterilization of the collection container (2) for the next specimen collection process. After the cleaning algorithm (B) is started by control unit (5) (201), disinfectant liquid stored into the tank (7) will be directed to the collection container (2) (202). The amount of disinfectant liquid will be determined according to the total amount of urea measured during the operation of working algorithm (A) (105). Because of the possibility of splashing of urea to the higher part of the collection container(2), disinfectant liquid more than the amount of total urea measured in a predetermined ratio will be directed to inside of the collection container (2) from the tank (7) (202). In order to clean the bacteria, viruses and fungi residues which possibly found into the urine, alcohol or derivatives can be used as disinfectant liquid.

After the transfer of the disinfectant liquid into the collection container (2) process, (202), the weight of the collection container (2) will be measured (203). Thus, it will be determined the volume of disinfectant liquid taken into the collection container (2) since the weight density of the disinfectant liquid is known.

Then, for the discharge of the disinfectant, pump (12) will be operated first (204). Thus, sterilization of the draining pipe (4) will be also performed with the help of the pump (12). Disinfectant located into the collection container (2) will be discharged into the general drain (11) with the help of the pump (12). Then, the valve (3) located beneath of the collection container (2) remaining urea in the lower part of the collection container (2) will be discharged into general drain (11) and valve (3) will be closed after waiting for predetermined period (206).

Then, in order to clean the urine specimen collection system (1) and purification of it from the disinfectant liquid, cleaning liquid will be directed into collection container (206). Required amount of cleaning liquid will be determined according to total amount of urea measured during the operation of working algorithm (A) (105). Because of the possibility of splashing of urea to the higher part of the collection container (2), cleaning liquid more than the amount of total urea measured in a predetermined ratio will be directed to inside of the collection container (2) (206). After these step, the weight of the collection container (2) will be measured (207). Then, for the discharge of the cleaning liquid, pump (12) will be operated first (208). Thus, cleaning of the draining pipe (4) in which urea and disinfectant liquid passes inside, will be also performed with the help of the pump (12). Cleaning liquid located into the collection container (2) will be discharged into the general drain (11) with the help of the pump (12) (208). Then, the valve (3) located beneath of the collection container (2), remaining urea in the lower part of the collection container (2) will be discharged into general drain (11) (209). After this step, it will be allowed that the weight of the collection container (29 will be equal to the weight of the empty collection container (2) (210). Thus, it is ensured that the collection container (2) is totally empty. After the complete discharge of the container (2), valve (3) will be closed after waiting for predetermined period (206).

The weight of the collection container (2) filled with the cleaning liquid will be measured in order to perform the urine specimen collection system's (1) own calibration (207). The weight of collection container (2) filled with cleaning liquid measured in weight measurement step (207) and the known weight of the cleaning liquid filling into the collection container (2) will be compared by control unit (5) (211). If the difference is bigger than the predetermined value, urine specimen collection system (1) is determined that the difference is due to the miscalculation performed by the load cell (6), give a warning and shut it down. If the difference measured is lower than the predetermined value, the system assumed that the difference is due to a dynamically change and the value will be used for calibration. Calibration of the urine specimen collection system (1) will be performed by control unit (5) and will be finalized by cleaning algorithm (212).

In the pumped version of the urine specimen collection system (1), urea, disinfectant liquid or cleaning liquid filled into the collection container (2) for cleaning and measurement is withdrawn by draining pipe (4) with the help of the pump (12). After this discharge, urea, disinfectant liquid or cleaning liquid is transported into the general drain (11). Urea remaining in the lower part of the collection container (2), is transported into specimen container (8) through the valve (3) located beneath the collection container. After the transfer of the urine, removal of specimen container (8) from inside of the urine specimen collection system (1) and beginning the operation of cleaning algorithm (B), the remaining disinfectant liquid or cleaning liquid into the collection container (2) is transported to general drain (11) through the opening of the valve (3) located beneath the collection container (2).

In the valved drain version of the urine specimen collection system (1), urea, disinfectant liquid or cleaning liquid filled into the collection container (2) for cleaning and measurement is withdrawn by draining pipe (4) with the help of the pump (12). During this discharge, the urea withdrawn from the collection container (2) first fills the specimen container (8) for a determined period and transported to general drain (11) by the pump (12) by the operation of a time dependent valve (13). Urea, disinfectant liquid and cleaning liquid remaining into the collection container (2) is directed to general drain (11) through the valve (3) located beneath the collection container (2).

In the single drain version of the urine specimen collection system (1), the urine collected from the patient for analysis collected into the collection container (2), is filled the specimen container(8) thorough the opening of the valve (3) located beneath the collection container (2) for a predetermined period. Then, after the removal of the specimen container(8) from the urine specimen collection system (1), by the opening of the valve(3) located beneath the collection container (2) by the control unit (5), remaining urea left in the collection container (2), can be discharged to the general drain (11). Thus, after the filling of the disinfectant liquid or cleaning liquid into the collection container (2), they are transferred to general drain (11) by the opening of the valve (3). In order to perform liquid transfers (cleaning and/or urea) to general drain when the valve (3) is open, funnel can be placed on the general drain (11).

Present invention can be used for the determination of the patients urination speed without collecting the specimens by replacing the step of opening of the valve (3) for the transfer of urea into the specimen container (8) (108) with opening of the valve (3) for the transfer of general drain (11) in the working algorithm (A) of the present invention. Then, there will be general drain (11) in place of specimen container (8) beneath the valve (3) located under the collection container (2).

Hereinabove, it is mentioned that the urine specimen collection system (1) can made audio and/or optic warnings to warn the patients. In order to monitor aforementioned warnings on the system, a touchscreen can be used. Aforementioned screen can possibly include the display of the graphics entire measurement period and information and warnings explaining operation of the system. A printer in the vicinity of the system or on the system can be used for reporting the results of the measurements to the patient.

Besides the information can be recorded as anonymous if the patient demands the related report as an anonymous user, if the patient demands the report by signing in his/her name, the related information can be made reachable to the patient and his/her physician.

In addition to aforementioned, diabetes can also be diagnosed over urea. Besides the urination speed measurements and urine density measurements, it is also possible to measure glucose values of the patients by employing the urine specimen collection system (1). Required urea specimen for these aforementioned measurements can be taken from inside the collection container (2) or draining pipe (4) by employing a receiver.

It is mentioned here that the present invention can be touch-operated for performing the aforementioned interactions. It is possible that patients may have concerns about touching the surface because the place of use of the present invention. Thus, it can be possible that by employing a proximity sensor (proximity switch) rather than touch-screen or button, the patient can control the properties of the system such as initiation of the system, shut down the system, and request the printed document, without touching the system. For alleviation of the high costs of loss of the printed documents and printer and screen, it can be possible that all the information can be transferred to patients mobile devices and patients can use these mobile devices. Mapping of the software to be loaded into the mobile device and urine specimen collection system (1) can be done over QR code, NFC, Bluetooth, WiFi. After the mapping, patient can govern all the functions of the urine specimen collection system (1) and download all the outputs generated by this process into his/her mobile device. The urine specimen collection system (1) can be initiated via barcodes already in use in hospitals for attaching onto the test specimens collected from patients. A barcode scanner can be placed onto the urine specimen collection system (1) for this purpose. After this aforementioned initiation, by recording the test result onto the barcode, it will become possible that all the information can be recorded into the hospital automation system with this number.

1. A urine specimen collection system, wherein a control unit executes a working algorithm for a usage during a urination of a person, wherein the working algorithm consisting of following steps:
measuring a weight of a collection container,
closing a valve in a proximity of lower part of the collection container in order to perform measurements on the collected urine,
measuring a weight of urine produced by patients in the collection container by a load cell,
recording difference between first and final weights and time dependent weight change of the collection container for a purpose to determine an amount of urine present in the collection container,
performing an operation of a pump for a discharge of urine,
performing an operation of the valve located at a lower part of the collection container for the discharge of the remaining urine left in the collection container;
opening a valve in the working algorithm for transferring the urine into a specimen container to collect a urine specimen from the urine transferred from a patient into the collection container.

2. (canceled)

3. The urine specimen collection system according to claim 1 wherein, the system further consists of a proximity sensor for interacting with the patient without touching.

4. The urine specimen collection system according to claim 1, wherein a cleaning algorithm for a sterilization and a cleaning of the urine specimen collection system for use of a second patient after an execution of working algorithm, wherein the cleaning algorithm comprises:
transferring disinfectant liquid more than the amount of total urine measured previously in a predetermined ratio directed to inside of the collection container from a tank,
discharging disinfectant liquid inside the collection container to a general drain by the operation of the pump,
discharging of the disinfectant liquid remaining in a lower part of the collection container by opening of the valve and closing the valve,
transferring cleaning liquid more than the amount of total urine measured previously in a predetermined ratio directed to inside of the collection container,
discharging the cleaning liquid inside the collection container to the general drain by operating the pump,
discharging the cleaning liquid remaining in the lower part of the collection container by opening of the valve,
waiting until the weight of the collection container is the same as an empty collection container.

5. The urine specimen collection system according to claim 4 wherein, the sterilization is performed by using municipal water supply rather than disinfectant liquid supplied from the tank.

6. The urine specimen collection system according to claim 4, wherein, the cleaning algorithm further comprises a step comparing the weight of the collection container filled with cleaning liquid and empty weight of the cleaning container, for the control of proper functioning of the load cell.

7. An urine specimen collection system for collecting a urine specimen and performing a urination speed test, the urine specimen collection system comprising;
a collection container, a valve, a load cell, a control unit wherein the control unit records a weight information from the load cell and determines an opening time of the valve and a specimen container;
the collection container into which the urine coming from a patient urinating into a reservoir is directed and the load cell measures a weight of the collection container dynamically and records these measurement values to the control unit,
a pump, wherein the pump is activated by the control unit when urine flow is ceased, in order to withdraw the urine collected into the collection container with a draining pipe to general drain; and
the valve located beneath the collection container, wherein the valve allows a transfer of the remaining urine which is not withdrawn by the draining pipe into the specimen container.
